(19) United States
(12) Patent Application Publication
(10) Pub. No.: US 2016/0068799 A1
(43) Pub. Date: Mar. 10, 2016

(54) MEDIUM, METHOD AND SYSTEM FOR CULTIVATION OF CHLORELLA PYRENOIDOSA OR ORGANISMS DERIVED FROM CHLORELLA PYRENOIDOSA

(71) Applicant: City University of Hong Kong, Kowloon Tong (HK)

(72) Inventors: Daniel PLEISSNER, Prenzlau (DE); Carol Sze Ki LIN, New Territories (HK)

(21) Appl. No.: 14/477,954

(22) Filed: Sep. 5, 2014

(51) Int. Cl. C12N 1/12

(52) U.S. Cl. CPC C12N 1/12

(57) ABSTRACT

The present invention is concerned a method for cultivation of Chlorella pyrenoidosa or organisms derived from Chlorella pyrenoidosa, comprising steps of a) providing a fermenter containing primarily a food waste hydrolysate medium, b) at the beginning of a first phase, introducing a strain of Chlorella pyrenoidosa or Chlorella pyrenoidosa derived organisms in the food waste hydrolysate medium for cultivation, wherein the food waste hydrolysate medium in the first phase contains substantially 5-30 g L⁻¹ glucose, 74-144 mg L⁻¹ free amino nitrogen and 40-64 mg L⁻¹ phosphate, c) during the first phase, allowing diminishing of free amino nitrogen and/or phosphate in the food waste hydrolysate medium until the free amino nitrogen and/or phosphate becomes limited, and d) at the beginning of a second phase after the first phase, feeding a fresh supply of food waste hydrolysate medium in the fermenter, wherein the fresh supply of food waste hydrolysate contains substantially 30-90 g L⁻¹ glucose, 160-230 mg L⁻¹ free amino nitrogen and 80-270 mg L⁻¹ phosphate, and feeding occurs at a certain dilution rate.

### FIELD OF THE INVENTION

The present invention is concerned with a medium, a method and a system for cultivation of Chlorella pyrenoidosa or organisms derived from Chlorella pyrenoidosa.

### BACKGROUND OF THE INVENTION

There is a variety of fermentation systems for use in treatment of food wastes. While these systems are useful in different extent or workable in converting food wastes into useful products, many of them are not very effective. The present invention proposes a useful alternative for treating food wastes. For example, the present invention suggests a medium, a method and a system for cultivation of Chlorella pyrenoidosa or organisms derived from Chlorella pyrenoidosa.

### SUMMARY OF THE INVENTION

According to a first aspect of the present invention, there is provided a method for cultivation of Chlorella pyrenoidosa or organisms derived from Chlorella pyrenoidosa, comprising steps of a) providing a fermenter containing primarily a food waste hydrolysate medium, b) at the beginning of a first phase, introducing a strain of Chlorella pyrenoidosa or Chlorella pyrenoidosa derived organisms in the food waste hydrolysate medium for cultivation, wherein the food waste hydrolysate medium in the first phase contains substantially 5-30 g L⁻¹ glucose, 74-144 mg L⁻¹ free amino nitrogen and 40-64 mg L⁻¹ phosphate, c) during the first phase, allowing diminishing of free amino nitrogen and/or phosphate in the food waste hydrolysate medium until the free amino nitrogen and/or phosphate becomes limited, and d) at the beginning of a second phase after the first phase, feeding a food waste hydrolysate medium in the fermenter, wherein the fresh food waste hydrolysate contains substantially 30-90 g L⁻¹ glucose, 160-230 mg L⁻¹ free amino nitrogen and 80-270 mg L⁻¹ phosphate, and feeding occurs at a certain dilution rate. During the first phase, there may be a step of maintaining the medium at a pH of substantially 6.5, maintaining a temperature of substantially 28° C., maintaining dissolved oxygen concentration in the medium of 20% or above, and/or stirring the medium at 400-800 rpm. The method map comprising, during the second phase, a step of maintaining the medium at a pH of substantially 6.5, maintaining a temperature of substantially 28° C., maintaining dissolved oxygen concentration in the medium at 20% or above, and/or stirring the medium at 400-800 rpm.

Preferably, the food waste hydrolysate medium in the first phase may contain substantially 0.1-0.5 g L⁻¹ fructose as an additional carbon source.

Suitably, the food waste hydrolysate medium in the second phase may contain substantially 0.8-2 g L⁻¹ fructose as an additional carbon source.

In one embodiment, the strain of Chlorella pyrenoidosa may be Chlorella pyrenoidosa supplied by Carolina Biological Supply Company.

Advantageously, the strain may be Chlorella pyrenoidosa.

In an embodiment, the step d) described above may be configured by hydrolysate composition and dilution rate to increase weight specific content of carbohydrates, proteins, lipids, saturated fatty acids, unsaturated fatty acids, alpha-linolenic acid, linoleic acid, oleic acid, stearic acid and palmitic acid by factors of substantially 1.76-1.11, 0.64-0.57, 2.54-3.05, 1.85-2.96, 2.42-3.17, 2.38-2.02, 1.89-2.98, 3.45-4.56, 2.42-5.57 and 1.56-2.21, respectively.

In one embodiment, the method may include provision of a reservoir for containing food waste hydrolysate medium, the food waste hydrolysate medium may be supplied from the reservoir to the fermenter during the second phase. The food waste hydrolysate medium may be supplied continuously from the reservoir to the fermenter during the second phase. The method may include provision of means for pumping the food waste hydrolysate medium from the reservoir to the fermenter during the second phase.

The method may also include provision of a harvest container for receiving culture broth from the fermenter, the culture broth containing biomass with enriched lipid, unsaturated- and saturated fatty acids contents, and biomass with reduced protein content.

The method may also include provision of means for pumping the culture from the fermenter to the harvest container.

### BRIEF DESCRIPTION OF THE DRAWINGS

The present invention will now be described by way of examples. Some embodiments of the present invention will now be explained, with reference to the accompanied drawings, in which:

FIG. 1 is a schematic representation of part of a fermentation system comprising food waste hydrolysate reservoir (1), pump (2), pumping food waste hydrolysate at a certain flow rate (Fᵢₙ) into the fermenter, fermenter (3), pump (4), pumping culture broth at a certain flow rate (Fₒᵤₜ) out of the fermenter and into a harvesting container (5).

FIG. 2 is a graph showing relationship of amount of different nutrients and biomass composition over time. Key: (A) biomass (open quadrate), glucose (closed circle) and fructose (open star) concentrations over time, (B) free amino nitrogen (FAN, closed quadrate) and phosphate (open circle) concentrations over time, (C) weight specific content of carbohydrates (closed quadrate), lipids (closed triangle) and proteins (open circle) and (D) weight specific contents of palmitic (open circle), stearic (closed quadrate), oleic (open quadrate), linoleic (closed triangle) and alpha-linolenic (closed star) acids for a continuous flow culture of Chlorella pyrenoidosa grown in food waste hydrolysate under nitrogen and/or phosphorous limitation(s) at a dilution rate of 0.91 day⁻¹.

FIG. 3 is a graph showing relationship of amount of different nutrients and biomass composition over time. Key: (A) biomass (open quadrate), glucose (closed circle) and fructose (open star) concentrations over time, (B) free amino nitrogen (FAN, closed quadrate) and phosphate (open circle) concentrations over time, (C) weight specific content of carbohydrates (closed quadrate), lipids (closed triangle) and proteins (open circle) and (D) weight specific contents of palmitic (open circle), stearic (closed quadrate), oleic (open quadrate), linoleic (closed triangle) and alpha-linolenic (closed star) acids for a continuous flow culture of Chlorella pyrenoidosa grown in food waste hydrolysate under nitrogen and/or phosphorous limitation(s) at a dilution rate of 0.36 day⁻¹.

FIG. 4 is a graph showing relationship of amount of different nutrients and biomass composition over time. Key: (A) biomass (open quadrate), glucose (closed circle) and fructose (open star) concentrations over time, (B) free amino nitrogen (FAN, closed quadrate) and phosphate (open circle) concentrations over time, (C) weight specific content of carbohydrates (closed quadrate), lipids (closed triangle) and proteins (open circle) and (D) weight specific contents of palmitic (open circle), stearic (closed quadrate), oleic (open quadrate), linoleic (closed triangle) and alpha-linolenic (closed star) acids for a batch culture of Chlorella pyrenoidosa grown in food waste hydrolysate under nutrient sufficiency.

### DETAILED DESCRIPTION OF PREFERRED EMBODIMENTS OF THE PRESENT INVENTION

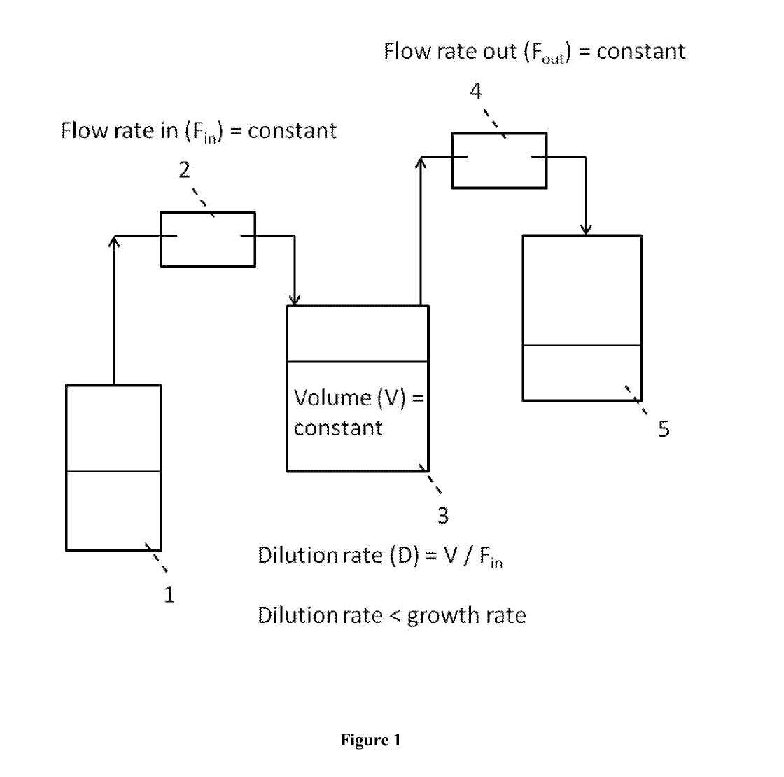

One aspect of the present invention is concerned with continuous flow culture system, performed using a fermentation apparatus shown in FIG. 1. In this particular embodiment, the microalga Chlorella pyrenoidosa is used. The fermentation is performed under nitrogen and/or phosphorous limitation(s) in food waste hydrolysate for production of biomass with increased carbohydrate, lipid and fatty acid contents, and decreased protein content. In other words, this system is configured to process food waste to produce useful algal biomass with higher contents of carbohydrate, lipid and fatty acid, and lower content of protein.

Referring to FIG. 1, it is shown the fermentative apparatus comprising of a food waste hydrolysate reservoir (1), a pump (2) for pumping food waste hydrolysate continuously at a certain flow rate (Fᵢₙ) into the fermenter (3) and a pump (4) pumping culture broth from the fermenter (3) at a certain flow rate (Fₒᵤₜ) into a harvesting container (5).

Three experiments were performed to illustrate workability of the present invention. In these experiments, food waste hydrolysate was prepared by enzymatic hydrolysis of mixed food waste consisting of noodles, rice, meat, eggs, bread, cake, vegetables and sauce. In order to control the nutrient composition of food waste hydrolysates being used in experiments, 780 g food waste (230 g dry weight) was hydrolyzed for 24 hours at 55° C. and pH 4-4.5 (uncontrolled), in a stirred fermenter (not shown) with either commercial glycolytic or proteolytic enzymes. The digestion of food waste by glycolytic enzymes resulted in a glucose rich and free amino nitrogen poor hydrolysate (122.3 g L⁻¹ glucose, 0.4 g L⁻¹ fructose, 240.0 mg L⁻¹ free amino nitrogen and 171.3 mg L⁻¹ phosphate), while the digestion of food waste by proteolytic enzymes resulted in a glucose poor and free amino nitrogen rich hydrolysate (30.7 g L⁻¹ glucose, 0.7 g L⁻¹ fructose, 990.0 mg L⁻¹ free amino nitrogen and 29.6 mg L⁻¹ phosphate). For experiments, both hydrolysates were mixed in an appropriate ratio and diluted with water to produce a fermentation feedstock with proper concentration of nutrients for culturing Chlorella pyrenoidosa in initial batch culture and continuous flow culture at a certain nutrient limitation.

At the end of the experiments, quantification of carbohydrate, lipid, protein and fatty acid contents was carried out in duplicate.

Experiment 1

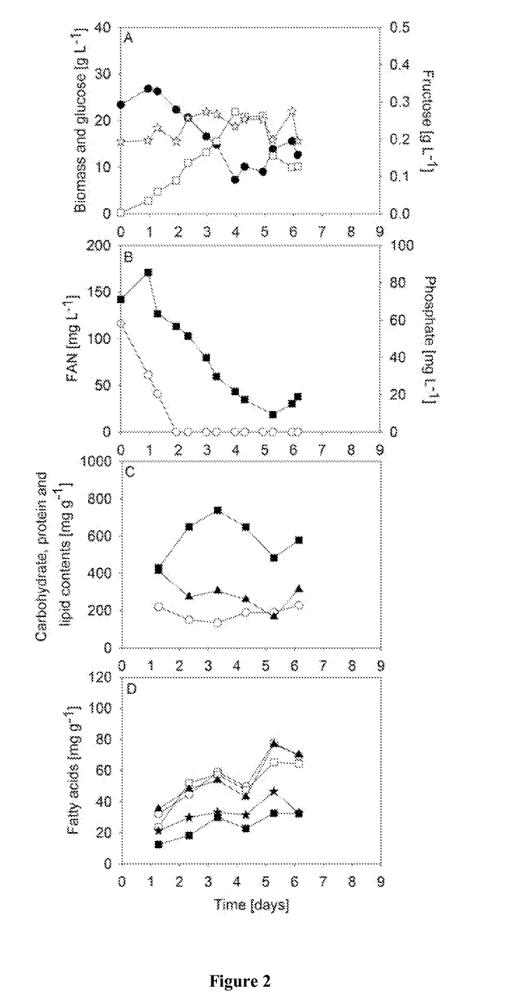

This experiment is shown in FIG. 2 and demonstrates the effects of nitrogen and/or phosphorous limitation(s) on Chlorella pyrenoidosa biomass produced in continuous flow culture in food waste hydrolysate at a dilution rate of 0.91 day⁻¹ with glucose as major carbon source, fructose as additional carbon source, free amino nitrogen as nitrogen source and phosphate as phosphorous source.

This experiment made use of an initial batch culture and a continuous flow culture. The food waste hydrolysate used for the initial batch culture contained 23.4 g L⁻¹ glucose, 0.2 g L⁻¹ fructose, 60 mg L⁻¹ phosphate and 142.1 mg L⁻¹ free amino nitrogen. The food waste hydrolysate used for the continuous flow culture contained 31.4 g L⁻¹ glucose, 0.8 g L⁻¹ fructose, 80 mg L⁻¹ phosphate and 166.2 mg L⁻¹ free amino nitrogen. Before use, the pH of each hydrolysate was adjusted to 6.5 by the addition of NaOH and then sterile filtered using a membrane filter (0.22 μm).

For this experiment, a strain of Chlorella pyrenoidosa available from the Carolina Biological Supply Company and identified by the number 15-2070 was used.

This experiment was performed in a fermentation apparatus that is shown in FIG. 1. The apparatus included a 2.5 L fermenter (3) of known type. The fermenter (3) was provided with a pH electrode (not shown) that was positioned in the fermenter (3) for monitoring the pH of the food waste hydrolysate and culture broth in the fermenter (3). The pH electrode (not shown) was connected via a control device to a pump (not shown). The control device was programmed to cause the pump (not shown) to pump 2 M NaOH or 2 M HCl from a reservoir (not shown) into the fermenter (3) when the pH electrode (not shown) detected a pH of the food waste hydrolysate and culture broth lower or greater than the predetermined value of 6.5. The fermenter (3) was also provided with a thermometer (not shown) and a heater (not shown) for maintaining the temperature of food waste hydrolysate and Chlorella pyrenoidosa culture broth at a desired temperature of 28° C. The fermenter (3) was also provided with an aerator (not shown) that was connected to a source of air (not shown) for aerating the food waste hydrolysate and culture broth within the fermenter (3) at a dissolved oxygen concentration above 20%. A dissolved oxygen sensor (not shown) was positioned for measuring dissolved oxygen concentration in the food waste hydrolysate and culture broth within the fermenter (3). A stirrer (not shown) was used for mixing the food waste hydrolysate and Chlorella pyrenoidosa culture broth.

Dissolved oxygen concentration was controlled by varying the stirrer speed (this was done manually). During initial batch culture and continuous flow culture, stirrer speed was set to 400-800 rpm.

For the initial batch culture, 1 L of food waste hydrolysate was filled into the fermenter (3). A 5% (v/v) inoculum of Chlorella pyrenoidosa, grown for 5 days in an orbital shaker at 28° C. and an initial pH of 6.5 in food waste hydrolysate similar in nutrient concentrations to the one used in initial batch phase was added to the fermenter (3). After inoculation, temperature, pH and dissolved oxygen concentration were adjusted and the cultivation started.

During the initial batch culture, samples of the culture were taken at regular time intervals for quantification of biomass, glucose, fructose, free amino nitrogen and phosphate concentrations, as such as weight specific contents of carbohydrates, lipids, proteins and fatty acids in Chlorella pyrenoidosa biomass.

During the initial batch culture, biomass concentration increased from 0.2 g L⁻¹ to 7.2 g L⁻¹ under consumption of glucose, free amino nitrogen and phosphate (FIGS. 2A and B). After 2 days, phosphate was depleted and the initial batch culture was changed to a continuous flow culture by adding food waste hydrolysate from a reservoir (1) at a dilution rate (D) of 0.91 days⁻¹ into the fermenter (3) using the pump (2). Growth rate (p) of Chlorella pyrenoidosa in initial batch culture was 1.4 days⁻¹. In order to avoid a wash-out of biomass and to establish nutrient limitation the dilution occurred at a rate below growth rate (D=0.91 days⁻¹, μ=1.4 days⁻¹, D<μ). Dilution rate was controlled by pump (2) pumping food waste hydrolysate from the reservoir (1) into the fermenter (3) at a certain flow rate (Fᵢₙ). At the same time, in order to maintain a constant volume of food waste hydrolysate inside the fermenter (3) culture broth was taken out by pump (4) at a certain flow rate (Fₒᵤₜ). The withdrawn culture broth was stored in a harvesting container (5) (FIGS. 1 and 2).

While phosphate was depleted after 2 days, free amino nitrogen was not and a steady decrease in free amino nitrogen concentration occurred until Day 5 (FIG. 2B). Free amino nitrogen was not consumed completely, but a remaining concentration of <20 mg L⁻¹ indicated that Chlorella pyrenoidosa was exposed to a dual limitation in nitrogen and phosphate from Days 5 to 6.

Biomass concentration increased despite dilution from 7.2 g L⁻¹ at Day 2 to more than 20 g L⁻¹ at Day 4, and washed out thereafter to around 10 g L⁻¹ at Day 6 (FIG. 2A).

Carbohydrate, lipid and protein contents showed fluctuations during initial batch and continuous flow cultures (FIG. 2C). Obviously, from the point where Chlorella pyrenoidosa experienced a limitation in phosphorous (Days 1-2) the weight specific contents of fatty acids increased, and further increased when nitrogen became limited (Days 4-5, FIG. 2D, Table 1). The continuous flow culture was stopped after 6 days.

Fructose was present in food waste hydrolysate as additional carbon source but was not consumed due to the presence of glucose (FIG. 2A).

Experiment 2

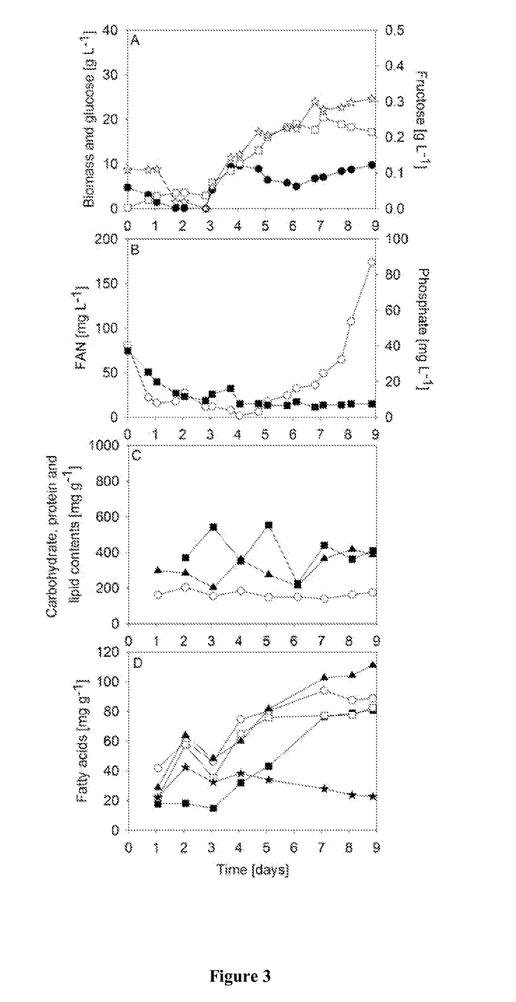

This experiment is shown in FIG. 3 and demonstrates the effects of nitrogen and/or phosphorous limitation(s) on Chlorella pyrenoidosa biomass produced in continuous flow culture at a dilution rate of 0.36 day⁻¹ with glucose as a major carbon source, fructose as additional carbon source, free amino nitrogen as nitrogen source and phosphate as phosphorous source.

This experiment made use of an initial batch culture and a continuous flow culture. The food waste hydrolysate used for the initial batch culture contained 4.7 g L⁻¹ glucose, 0.1 g L⁻¹ fructose, 40.5 mg L⁻¹ phosphate and 74.3 mg L⁻¹ free amino nitrogen. The food waste hydrolysate used for the continuous flow culture contained 89.0 g L⁻¹ glucose, 1.9 g L⁻¹ fructose, 272.2 mg L⁻¹ phosphate and 227.0 mg L⁻¹ free amino nitrogen. Before use, the pH of each hydrolysate was adjusted to 6.5 by the addition of NaOH and the food waste hydrolysate was then sterile filtered using a membrane filter (0.22 μm).

For this experiment, a strain of Chlorella pyrenoidosa available from the Carolina Biological Supply Company and identified by the number 15-2070.

This experiment was performed in a fermentation apparatus that is shown in FIG. 1. The apparatus included a 2.5 L fermenter (3) of known type. The fermenter (3) was provided with a pH electrode (not shown) that was positioned in the fermenter (3) for monitoring the pH of the food waste hydrolysate and culture broth in the fermenter (3).

The pH electrode (not shown) was connected via a control device to a pump (not shown). The control device was programmed to cause the pump (not shown) to pump 2 M NaOH or 2 M HCl from a reservoir (not shown) into the fermenter (3) when the pH electrode (not shown) detected a pH of the food waste hydrolysate and culture broth lower or greater than the predetermined value of 6.5. The fermenter (3) was also provided with a thermometer (not shown) and a heater (not shown) for maintaining the temperature of food waste hydrolysate and Chlorella pyrenoidosa culture broth at a desired temperature of 28° C. The fermenter (3) was also provided with an aerator (not shown) that was connected to a source of air (not shown) for aerating the food waste hydrolysate and culture broth within the fermenter (3) at a dissolved oxygen concentration above 20%. A dissolved oxygen sensor (not shown) was positioned for measuring dissolved oxygen concentration in the food waste hydrolysate and culture broth within the fermenter (3). A stirrer (not shown) was used for mixing the food waste hydrolysate and Chlorella pyrenoidosa culture broth. Dissolved oxygen concentration was controlled by varying the stirrer speed (this was done manually). During initial batch culture and continuous flow culture stirrer speed was set to 400-800 rpm.

For initial batch culture, 1 L of food waste hydrolysate was filled into the fermenter (3). A 5% (v/v) inoculum of Chlorella pyrenoidosa, grown for 5 days in an orbital shaker at 28° C. and an initial pH of 6.5 in food waste hydrolysate similar in nutrient concentrations to the one used in initial batch phase, was added to the fermenter (3). After inoculation, temperature, pH and dissolved oxygen concentration were adjusted and the cultivation started.

During the initial batch culture, samples of the culture were taken at regular time intervals for quantification of biomass, glucose, fructose, free amino nitrogen and phosphate concentrations, as such as weight specific contents of carbohydrates, lipids, proteins and fatty acid contents in Chlorella pyrenoidosa biomass.

During initial batch culture, biomass concentration increased from 0.2 g L⁻¹ to 2.9 g L⁻¹ under consumption of glucose, free amino nitrogen and phosphate (FIGS. 3A and B). After 2.8 days, phosphorous and nitrogen (free amino nitrogen concentration<25 mg L⁻¹) became limited and the initial batch culture was changed to a continuous flow culture by adding food waste hydrolysate from a reservoir (1) at a dilution rate (D) of 0.36 days⁻¹ into the fermenter (3) using the pump (2). Growth rate (μ) of Chlorella pyrenoidosa in initial batch culture was 1.4 days⁻¹. In order to avoid a wash-out of biomass and to establish nutrient limitation the dilution occurred at a rate below growth rate (D=0.36 days⁻¹, p=1.4 days⁻¹, D<p). Dilution rate was controlled by pump (2) pumping food waste hydrolysate from the reservoir (1) into the fermenter (3) at a certain flow rate (Fᵢₙ). At the same time, in order to maintain a constant volume of food waste hydrolysate inside the fermenter (3) culture broth was taken out by pump (4) at a certain flow rate (Fₒᵤₜ). The withdrawn culture broth was stored in a harvesting container (5) (FIGS. 1 and 3).

From Day 5 the phosphate concentration increased steadily to more than 80 mg L⁻¹ on Day 9. The continuous flow culture was stopped after 8.9 days.

Fructose was present in food waste hydrolysate as additional carbon source but was not consumed due to the presence of glucose (FIG. 3A).

Carbohydrate, lipid and protein contents showed fluctuation during initial batch culture and continuous flow culture (FIG. 3C). Obviously, from the point where Chlorella pyrenoidosa experienced a dual limitation in phosphorous and nitrogen (Day 2-3) the weight specific contents of fatty acids increased 2 to 3 folds (FIG. 3D, Table 1).

Experiment 3

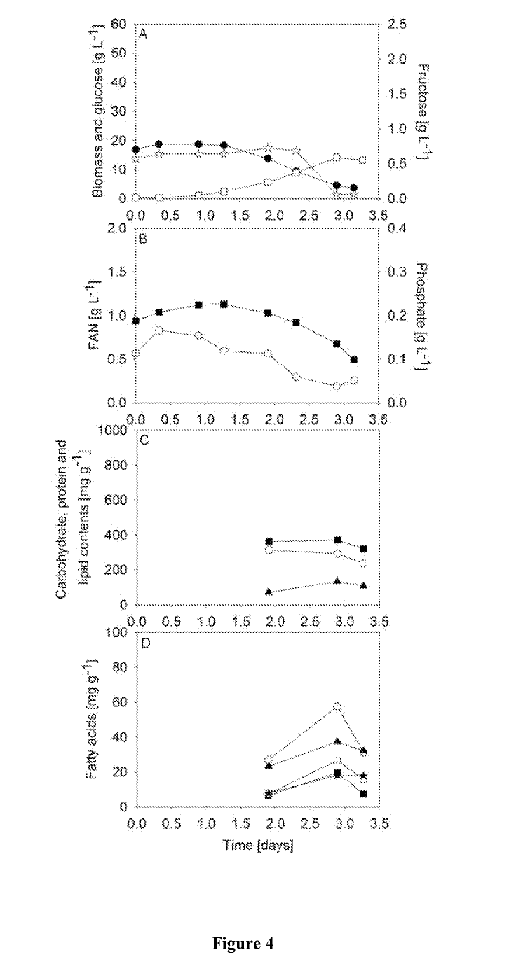

This experiment is shown in FIG. 4 and demonstrates the effect of nutrient sufficiency on Chlorella pyrenoidosa biomass produced in batch culture in food waste hydrolysate with glucose as a major carbon source, fructose as additional carbon source, free amino nitrogen as nitrogen source and phosphate as phosphorous source.

This experiment consists of a batch culture. The food waste hydrolysate used for the batch culture contained 16.0 g L⁻¹ glucose, 0.4 g L⁻¹ fructose, 96.0 mg L⁻¹ phosphate and 888.8 mg L⁻¹ free amino nitrogen. Before use, the pH of the hydrolysate was adjusted to 6.5 by the addition of NaOH and the food waste hydrolysate was then sterile filtered using a membrane filter (0.22 μm).

For this experiment, a strain of Chlorella pyrenoidosa available from the Carolina Biological Supply Company and identified by the number 15-2070.

This experiment was performed in a fermentation apparatus that is shown in FIG. 1, without pumps (2 and 4), food waste hydrolysate reservoir (1) and harvesting container (5). The apparatus included a 2.5 L fermenter (3) of known type.

The fermenter (3) was provided with a pH electrode (not shown) that was positioned in the fermenter (3) for monitoring the pH of the food waste hydrolysate and culture broth in the fermenter (3).

The pH electrode (not shown) was connected via a control device to a pump (not shown). The control device was programmed to cause the pump (not shown) to pump 2 M NaOH or 2 M HCl from a reservoir (not shown) into the fermenter (3) when the pH electrode (not shown) detected a pH of food waste hydrolysate and culture broth lower or greater than the predetermined value of 6.5. The fermenter (3) was also provided with a thermometer (not shown) and a heater (not shown) for maintaining the temperature of food waste hydrolysate and Chlorella pyrenoidosa culture broth at a desired temperature of 28° C. The fermenter (3) was also provided with an aerator (not shown) that was connected to a source of air (not shown) for aerating the food waste hydrolysate and culture broth within the fermenter (3) at a dissolved oxygen concentration above 20%. A dissolved oxygen sensor (not shown) was positioned for measuring dissolved oxygen concentration in the food waste hydrolysate and culture broth within the fermenter (3). A stirrer (not shown) was used for mixing the food waste hydrolysate and Chlorella pyrenoidosa culture broth. Dissolved oxygen concentration was controlled by varying the stirrer speed (this was done manually). Stirrer speed was set to 400-800 rpm.

For the batch culture, 1 L of food waste hydrolysate was filled into the fermenter (3). A 5% (v/v) inoculum of Chlorella pyrenoidosa, grown for 5 days in an orbital shaker at 28° C. and an initial pH of 6.5 in food waste hydrolysate similar in nutrient concentrations to the one added to the fermenter (3), used. After inoculation, temperature, pH and dissolved oxygen concentration were adjusted and the cultivation started.

During the batch culture, samples of the culture were taken at regular time intervals for determination of biomass, glucose, fructose, free amino nitrogen and phosphate concentrations, as such as weight specific contents of carbohydrates, lipids, proteins and fatty acid contents in Chlorella pyrenoidosa biomass.

During the batch culture, biomass concentration increased from 0.5 g L⁻¹ to 14.1 g L⁻¹ under consumption of glucose, free amino nitrogen and phosphate (FIG. 4). Glucose was the only nutrient that became obviously depleted after 3 days. Interestingly, when glucose was almost consumed Chlorella pyrenoidosa started to use fructose as an additional carbon source. Hence, fructose contributes to biomass and lipid formation. The batch culture was stopped after 3.3 days in order to avoid multiple nutrient limitations.

Carbohydrate, lipid and protein contents were constant during the batch cultures (FIG. 4C). Weight specific contents of fatty acids remained mostly constant and only the palmitic acid content showed a fluctuation during cultivation (FIG. 4D, Table 1).

Table 1 below shows the weight specific contents of biomass constituents of Chlorella pyrenoidosa cultured in batch culture (nutrient sufficiency) or continuous flow culture (nitrogen and/or phosphorous limitation(s).

TABLE 1
Weight specific contents (±S.D.) of biomass constituents in Chlorella pyrenoidosa
biomass produced in batch cultures under condition of nutrient sufficiency or in
continuous flow cultures at a dilution rate (D) of 0.91 or 0.36 day⁻¹ under
nitrogen and/or phosphorous limitation(s).

| | | Continuous | | Continuous | |
|---|---|---|---|---|---|
| | Batch | flow culture, | | flow culture, | |
| | culture | D = 0.91 day⁻¹ | | D = 0.36 day⁻¹ | |
| Carbohydrates [mg g⁻¹] | 351.7 ± 26.7 | 619.0 ± 94.5 | 1.76* | 390.9 ± 109.2 | 1.11* |
| Proteins [mg g⁻¹] | 281.4 ± 40.7 | 178.8 ± 37.1 | 0.64* | 160.0 ± 15.9 | 0.56* |
| Lipids [mg g⁻¹] | 103.8 ± 31.9 | 263.6 ± 58.9 | 2.54* | 317.4 ± 86.8 | 3.05* |
| Major saturated fatty | 49.7 ± 23.9 | 18.2 ± 19.8 | 1.85* | 147.4 ± 30.1 | 2.96* |
| acids [mg g⁻¹] | | | | | |
| Major unsaturated fatty | 62.0 ± 22.0 | 150.4 ± 27.5 | 2.42* | 196.9 ± 20.9 | 3.17* |
| acids [mg g⁻¹] | | | | | |
| Palmitic acid [mg g⁻¹] | 38.5 ± 16.6 | 60.1 ± 13.7 | 1.56* | 85.1 ± 7.6 | 2.21* |
| Stearic acid [mg g⁻¹] | 11.2 ± 7.3 | 27.1 ± 6.4 | 2.42* | 62.3 ± 22.9 | 5.57* |
| Oleic acid [mg g⁻¹] | 16.6 ± 9.6 | 57.3 ± 7.7 | 3.45* | 75.6 ± 6.5 | 4.56* |
| Linoleic acid [mg g⁻¹] | 30.8 ± 7.1 | 58.4 ± 14.5 | 1.89* | 91.9 ± 21.0 | 2.98* |
| Alpha-linolenic acid | 14.6 ± 5.9 | 34.7 ± 6.7 | 2.38* | 29.4 ± 6.6 | 2.02* |
| [mg g⁻¹] | | | | | |

*change in weight specific content of biomass constituent = continuous flow culture/batch culture

When compared to Chlorella pyrenoidosa biomass produced under nutrient sufficiency in food waste hydrolysate, the production of Chlorella pyrenoidosa biomass in continuous flow culture in food waste hydrolysate under nitrogen and/or phosphorous limitation(s) results in 2 to 5 times higher weight specific carbohydrate, lipid and fatty acid concentrations, while the protein content is reduced by around 50% due to nitrogen limitation.

The harvested, carbohydrate, lipid and fatty acid rich Chlorella pyrenoidosa biomass may be used as food additives, as feed in aquaculture, particularly for fish and shellfish larvae, and other marine animals, and as feed for poultry. The fatty acid profile may make the biomass interesting as a feedstock in biodiesel production and synthesis of chemicals (e.g. plasticizers and surfactants).

Other strains of Chlorella pyrenoidosa and other heterotrophic microalgae may also grow in food waste hydrolysate and may show similar response to nitrogen and/or phosphorous limitation(s). Also other hexoses and pentoses obtainable from food waste after hydrolysis may serve as carbon sources for heterotrophic microalgae.

It should be understood that certain features of the invention, which are, for clarity, described in the content of separate embodiments, may be provided in combination in a single embodiment. Conversely, various features of the invention which are, for brevity, described in the content of a single embodiment, may be provided separately or in any appropriate sub-combinations. It is to be noted that certain features of the embodiments are illustrated by way of non-limiting examples. Also, a skilled person in the art will be aware of the prior art which is not explained in the above for brevity purpose.

1. A method for cultivation of Chlorella pyrenoidosa or organisms derived from Chlorella pyrenoidosa, comprising steps of:
a) providing a fermenter containing primarily a food waste hydrolysate medium;
b) at the beginning of a first phase, introducing a strain of Chlorella pyrenoidosa or Chlorella pyrenoidosa derived organisms in the food waste hydrolysate medium for cultivation, wherein the food waste hydrolysate medium in the first phase contains substantially 5-30 g L⁻¹ glucose, 74-144 mg L⁻¹ free amino nitrogen and 40-64 mg L⁻¹ phosphate;
c) during the first phase, allowing diminishing of free amino nitrogen and/or phosphate in the food waste hydrolysate medium until the free amino nitrogen and/or phosphate becomes limited;
d) at the beginning of a second phase after the first phase, feeding a fresh food waste hydrolysate medium in the fermenter, wherein the fresh food waste hydrolysate contains substantially 30-90 g L⁻¹ glucose, 160-230 mg L⁻¹ free amino nitrogen and 80-270 mg L⁻¹ phosphate, and feeding occurs at a certain dilution rate.

2. A method as claimed in claim 1, wherein the food waste hydrolysate medium in the first phase contains substantially 0.1-0.5 g L⁻¹ fructose as an additional carbon source.

3. A method as claimed in claim 1, wherein the food waste hydrolysate medium in the second phase contains substantially 0.8-2 g L⁻¹ fructose as an additional carbon source.

4. A method as claimed in claim 1, comprising, during the second phase, a step of maintaining the medium at a pH of substantially 6.5, maintaining a temperature of substantially 28° C., maintaining dissolved oxygen concentration in the medium at 20% or above, and/or stirring the medium at 400-800 rpm.

5. A method as claimed in claim 1, wherein the strain of Chlorella pyrenoidosa is Chlorella pyrenoidosa supplied by Carolina Biological Supply Company.

6. A method as claimed in claim 1, wherein said step d) is configured to increase weight specific content of carbohydrates, proteins, lipids, saturated fatty acids, unsaturated fatty acids, alpha-linolenic acid, linoleic acid, oleic acid, stearic acid and palmitic acid by factors of substantially 1.76-1.11, 0.64-0.57, 2.54-3.05, 1.85-2.96, 2.42-3.17, 2.38-2.02, 1.89-2.98, 3.45-4.56, 2.42-5.57 and 1.56-2.21, respectively.

7. A method as claimed in claim 1, comprising provision of a reservoir for containing food water hydrolysate medium, the food waste hydrolysate medium is supplied from the reservoir to the fermenter during the second phase.

8. A method as claimed in claim 1, wherein the food waste hydrolysate medium is supplied continuously from the reservoir to the fermenter during the second phase.

9. A method as claimed in claim 1, comprising provision of means for pumping the food waste hydrolysate medium from the reservoir to the fermenter during the second phase.

10. A method as claimed in claim 1, comprising provision of a harvest container for receiving culture broth from the fermenter, the culture broth containing biomass with enriched lipid, unsaturated- and saturated fatty acids contents, and biomass with reduced protein content.

11. A method as claimed in claim 1, comprising provision of means for pumping the culture from the fermenter to the harvest container.
